(19) United States
(12) Patent Application Publication
(10) Pub. No.: US 2006/0253195 A1
(43) Pub. Date: Nov. 9, 2006

(54) Eye implant and methods of positioning an artifical eye implant with the eye of a human or an animal

(76) Inventor: Cesar C. Carriazo, Miami, FL (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(21) Appl. No.: 11/220,833

(22) Filed: Sep. 7, 2005

Related U.S. Application Data
(60) Provisional application No. 60/677,737, filed on May 4, 2005.

(30) Foreign Application Priority Data
May 4, 2005 (EP) 05 009 817.7

(51) Int. Cl. A61F 2/16; A61F 9/013

(52) U.S. Cl. 623/6.13; 606/107

(57) ABSTRACT

The invention relates to an artificial eye implant, especially as a substitute for an eye lens or a vitreous body of a human or animal eye consisting of a hollow body, wherein the hollow body is made of a biocompatible, flexible and extensible material and is able to be filled with a medium. The present invention further relates to methods of positioning an artificial eye implant within the eye of a human or an animal generally comprising the steps of: preparing an eye to receive an intraocular artificial eye implant wherein said implant consists of a hollow body made of a biocompatible, flexible and extensible material, said hollow body is able to be filled with a medium; inserting said intraocular eye implant into said eye via a small cut in said eye; positioning said implant in the non-filled state in the capsule bag or the posterior orbit of the eye; filling said implant with said medium; and closing the eye where said implant was inserted.

### CROSS-REFERENCE TO RELATED APPLICATIONS

This application claims convention priority from European patent application no. 05 009 817.7, with a filing date of May 4, 2005. This application also claims the benefit of U.S. provisional patent application No. 60/677,737, with a filing date of May 4, 2005 The specifications and drawings of European patent application no. 05 009 817.7 and of U.S. provisional patent application No. 60/677,737 are specifically hereby incorporated herein by reference.

### BACKGROUND OF THE INVENTION

The present invention relates to an artificial eye implant, especially as a substitute for an eye lens or a vitreous body of a human or animal eye consisting of a hollow body. The present invention further relates to methods of positioning an artificial eye implant within the eye of a human or an animal.

Today, the clouding of the eye lens referred to as cataract is a very frequent disease, by which especially elder people are afflicted. The only possibility of correction is the surgical treatment. It consists in removing the anterior lenticular capsule, lenticular cortex and lenticular nucleus with subsequent implantation of a so-called intraocular lens (IOL), which is introduced into the lenticular capsule bag, and may be made of different materials. Concerning the restoration of the visual acuity, very good successes are achieved with this approach. However, concerning the restoration of the normal accommodation function of the lens with the possibility of focusing in the near and far range without the need for correcting visual aids, the results of surgical interventions are still insufficient.

Today, in the surgical cataract treatment, there is worldwide the tendency to perform the intervention with local anesthesia and with increasingly smaller cuts. In this manner, the application of sutures can be omitted, which delay the restoration of the visual power and can result in formation of astigmatism. At the same time, a plurality of IOL types has been developed, which should allow for restoration of the accommodation ability. Various lens designs with small resilient retaining legs (so-called haptics) with different shape belong to that, which are to allow for central alignment within the capsule bag (e.g. DE 35 03 690 A1, DE 36 35 111 μl, US 4725277 A, US 4842602 A). However, the employment of these lens constructions did not produce satisfying results, since the formation of a capsule bag fibrosis restricts the positional change of the intraocular lens (IOL).

Also the development of different IOL with an optical zone for the near and far range as well as for the middle visual range has proven to be only partially successful, because the concerned IOL (e.g. DE 40 38 088 A1, US 5628798 A, US 2003/0149480 A1, US 2004/0148022 A1) are not capable of accommodation in the original meaning.

Since a few years, the fabrication of artificial lenses of extensible materials is also considered. Against this background, experiments with different lens types of flexible materials, with resilient filters and the possibility of adjusting the refractive power subsequent to the implantation by means of irradiation with light according to the patient, and of materials expanding depending on temperature (smart lens, e.g. US 2005/021139 A1) were performed. However, these lens constructions, which are to allow for insertion of the implant through very small cuts, are presently still tested for their biocompatibility with human tissue and for their behavior in the eye.

In the attempt to replace the human eye lens by artificial lenses of comparable size, up to now, the limiting factor is in the circumstance that large surgical cuts are required for this, whereas worldwide the opposite tendency of performing very small cuts is to observe. Also the approach of liquid injection directly into the capsule bag is limited mainly for two reasons. On the one hand, the capsule bag must not be opened during injection of the liquid. However, exactly this is always the case, since performing an anterior capsulorhexis (opening the anterior lenticular capsule) is required for the cataract extraction, so that the liquid would leak into the anterior ocular chamber. On the other hand, the injected liquids have to solidify within the eye in order to be able to perform lens functions. However, a series of chemical reactions are required for this, which cannot occur in the interior of the eye.

Silicone oil, which has similar characteristics as the vitreous body, is one of the liquids, which are most often used today, by which the retina is again fitted after retinal detachment. Since silicone oil has a higher density than water, it is used as a vitreous body substitute in patients with retinal detachment after the surgical removal of the vitreous body (vitrectomy), in order to fit again the retina in this manner. Silicone oil is often employed today, however, it has two great disadvantages. The first disadvantage results from the circumstance that, in case of existing retinal perforations, the silicone oil can penetrate behind the retina despite its high density, and in this manner cause again retinal detachment. The second disadvantage is in the fact that after contact with the liquids present in the interior of the eye for plural months, an emulgating process and retinal toxicity can occur, so that it becomes necessary to remove the silicone oil from the eye.

### SUMMARY OF THE INVENTION

Therefore, the object of the invention is to provide an eye implant of the initially mentioned type, which overcomes the mentioned disadvantages of conventional eye implants, and especially provides a substitute for an eye lens or a vitreous body of a human or animal eye capable of accommodation, wherein the insertion of the implant can be performed in minimally invasive manner.

This object is solved by a generic artificial eye implant having the features of claim 1.

Advantageous developments of the artificial eye implant are described in the dependent claims.

An artificial eye implant according to the invention, especially as a substitute for an eye lens or a vitreous body of a human or animal eye, is comprised of a hollow body, wherein the hollow body is made of a biocompatible, flexible and extensible material and is able to be filled with a medium. By such a development of the eye implant, it is ensured that it can be very easily attached within the eye in the non-filled state, so that a corresponding intervention on the human or animal eye can be performed in minimally invasive manner. Further, by the eye implant according to the invention, it is ensured that restoration of the accommodation ability of the patient is effected. By introducing the hollow body as a substitute for the eye lens and by filling out the entire capsule bag, the vibrosation of anterior capsule and posterior capsule can be avoided, so that the ciliary muscle of the eye can again perform its accommodation function. Since is serves for use of the artificial eye implant as a substitute for an eye lens, the hollow body is formed lens-shaped in the state filled with a medium, and has as a whole similar characteristics as the natural human or animal eye lens with respect to thickness, flexibility, refractive power and accommodation ability. Additionally, no attaching devices such as haptics are required for the eye implant according to the invention, since the eye implant fully occupies the position of the natural eye lens and thus is automatically fixed within the capsule bag. If the artificial eye implant according to the invention is used as a substitute for a vitreous body of the human or animal eye, thus the hollow body models approximately the shape of the vitreous body of the human or animal eye in a state filled with the medium. Thus, the eye implant according to the invention is filled with silicone oil or with another liquid after introduction into the orbit. Additionally, by the eye implant formed as a hollow body it is ensured that the introduced liquid cannot come into contact with the tissue structures and liquids present in the interior of the eye on the one hand, and cannot flow behind the retina on the other hand, since it is completely enclosed in the cavity. For this reason, advantageously, it is also not required to remove it later again from the eye.

In an advantageous development of the invention, the hollow body has at least one valve device for filling the cavity with the medium. Thereby, it is possible without complications to fill the hollow body with the appropriate medium, without the medium entering the surrounding eye tissue. The filling can for example be effected by a syringe filled with the medium and a corresponding cannula.

In another advantageous development, the hollow body has at least one tube connection for filling the medium into the cavity. The tube connection can for example be formed as a flexible micro-tube. Thus, advantageously, after introducing the eye implant into the capsule bag of the eye, the cavity can be filled with the medium in controlled manner through the micro-tube. After completing the filling operation, the tube can be removed again.

However, it is also possible that the hollow body of the artificial eye implant according to the invention has neither a valve device nor a tube connection for filling the cavity with the medium. Thus, the biocompatible material of the hollow body can be selected such that it automatically closes due to its elastic properties, for example if an injection needle is removed after the filling operation. The above mentioned tube connection can also be connected via the also mentioned valve device among other things, so that the hollow body also automatically closes after removing the tube connection.

In another advantageous development of the invention, the hollow body has at least one device with optical refractive power on its side facing away from the interior of the eye. Further, it is possible that the hollow body has at least one device with optical refractive power also or exclusively on its side facing the interior of the eye. By the corresponding development or modeling of the front and/or backside of the hollow body or eye implant, respectively, optical corrections can be performed in targeted manner depending on the patient needs. The refractive index of the medium can also be adapted to the patient needs.

In an advantageous development of the artificial eye implant according to the invention, the medium is liquid and/or semi-liquid and/or gaseous. Here, physiological liquids, silicone gels and oils, polymer compounds and noble gases are to be mentioned exemplarily. Basically, each material can be used as the medium, which on the one hand is suitable for filling the hollow body, and on the other hand has predefined physical properties such as for example the mentioned refractive index. Only media, which attack the material of the hollow body itself or react chemically with it, respectively, cannot be used as the medium.

In accordance with the objectives of the invention there are provided several methods of positioning an artificial eye implant within the eye of a human or an animal. In general a method of positioning an artificial eye implant within the eye of a human or an animal is provided comprising the steps of:
a) preparing an eye to receive an intraocular artificial eye implant wherein said implant consists of a hollow body made of a biocompatible, flexible and extensible material, said hollow body is able to be filled with a medium;
b) inserting said intraocular eye implant into said eye via a small cut in said eye;
c) positioning said implant in the non-filled state in the capsule bag or the posterior orbit of the eye;
d) filling said implant with said medium; and
e) closing the eye where said implant was inserted.

### BRIEF DESCRIPTION OF THE DRAWINGS

Further advantages, details and features of the eye implant according to the invention are exemplarily illustrated by way of several embodiments represented in the figures. There show

FIG. 1 a schematic representation of a human eye;

FIG. 2 a schematic representation of the human eye after removing the eye lens;

FIG. 3 a schematic representation of a human eye with an implanted artificial eye implant according to the prior art;

FIG. 4 a schematic representation of an inventive eye implant according to a first embodiment during introduction into the human eye;

FIG. 5 a schematic representation of the inventive eye implant according to FIG. 4 upon introduction into the lenticular capsule of the eye;

FIG. 6 a schematic representation of the inventive eye implant according to FIG. 4 with connected filling device;

FIG. 7 a schematic representation of the inventive eye implant according to FIG. 4 in a state filled with medium;

FIG. 8 a schematic representation of an inventive eye implant according to a second embodiment in a state filled with medium;

FIG. 9 a schematic representation of an inventive eye implant according to a third embodiment during introduction into a posterior orbit of the human eye;

FIG. 10 a schematic representation of an inventive eye implant according to FIG. 9 with connected filling device;

FIG. 11 a schematic representation of the inventive eye implant according to FIG. 9 in a state filled with medium; and

FIG. 12 a representation of an inventive eye implant according to a fourth embodiment.

### DETAILED DESCRIPTION OF PREFERRED EMBODIMENTS

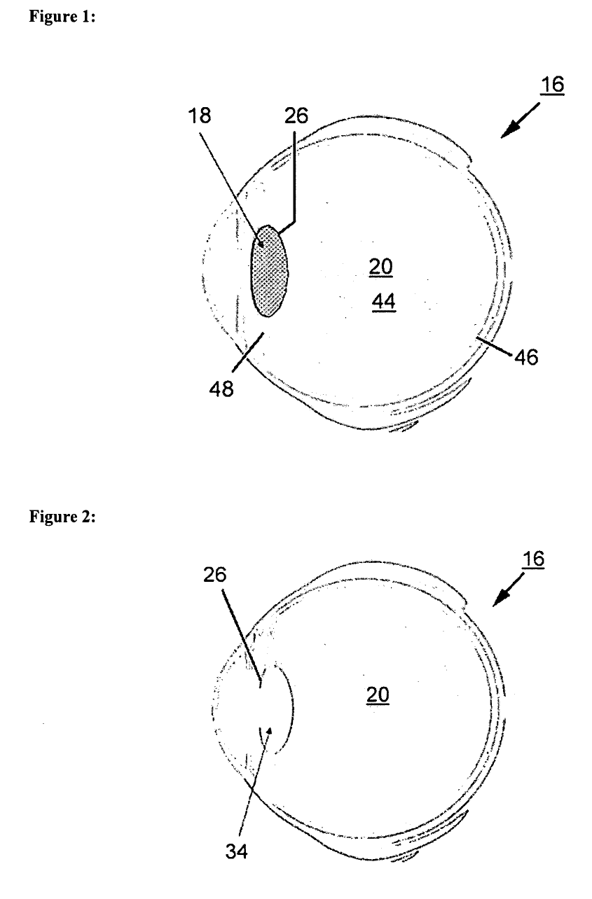

FIG. 1 shows a schematic representation of a human eye 16. One recognizes the position of the eye lens 18 with the ciliary muscles 48 required for accommodation of the lens 18. Therein, the eye lens 18 is supported in a lenticular capsule or a capsule bag 26, respectively. Further, the interior of the eye includes a vitreous body 20 within the vitreous body space 24 as well as the retina 46 surrounding the vitreous body space 24.

FIG. 2 shows a schematic representation of the human eye 16 after removing the eye lens 18. One recognizes that the capsule bag 26 is now opened and a cavity 34 forms within the capsule bag 26. Removal of the eye lens 18 can for example be required due to its clouding and thus the loss of the functionality.

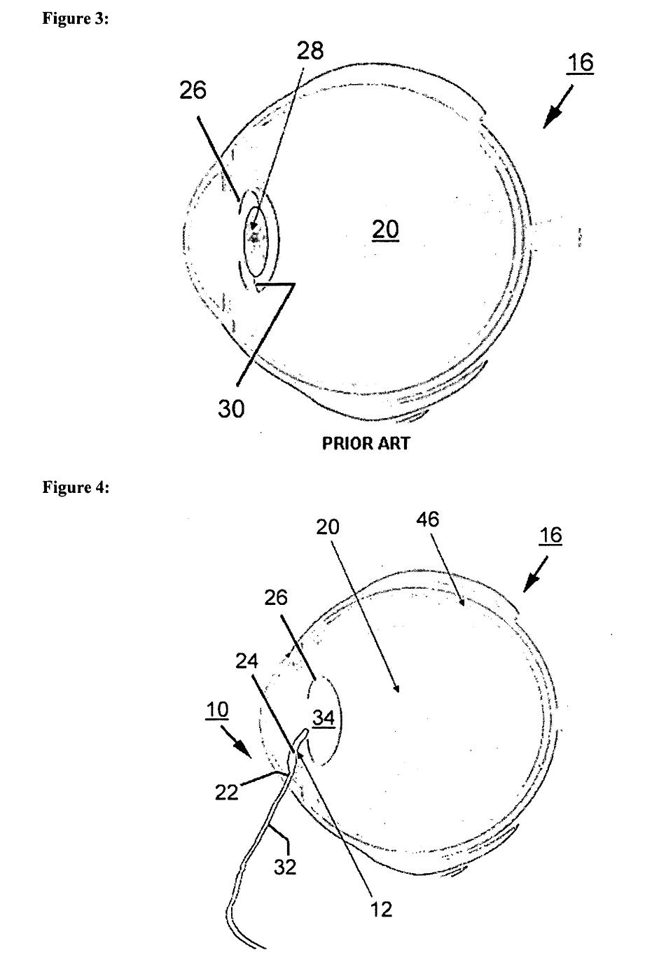

FIG. 3 shows a schematic representation of a human eye 16 with an implanted artificial eye implant 28 according to the prior art. One recognizes that the artificial eye implant has plural haptics 30 for positional fixation within the capsule bag 26. The disadvantages of such known eye implants have already been described above.

FIG. 4 shows a schematic representation of an artificial eye implant 10 according to a first embodiment of the invention. Therein, the representation shows the eye implant 10 during introduction into the human eye 16. Therein, the introduction is effected through a small cut already used for removing the eye lens 18. One recognizes that the eye implant 10 shown in the illustrated embodiment serves as a substitute for the eye lens and includes a hollow body 12. Therein, the hollow body 12 is made of a biocompatible, flexible and extensible material and is able to be filled with a medium 14. As the materials for the artificial eye implant, especially biocompatible, flexible and extensible plastics, silicones and a plurality of other polymer compounds are considered, which have the requested properties. Silicone gum, acrylate, polyurethane, latex and polymethylmethacrylate (PMMA) are to be mentioned exemplarily.

Further, one recognizes that the eye implant 10 has a valve device 22 for filling the cavity 24 with the medium 14. A tube connection 32 is detachably connected to the valve device 22, which in turn leads to a medium source.

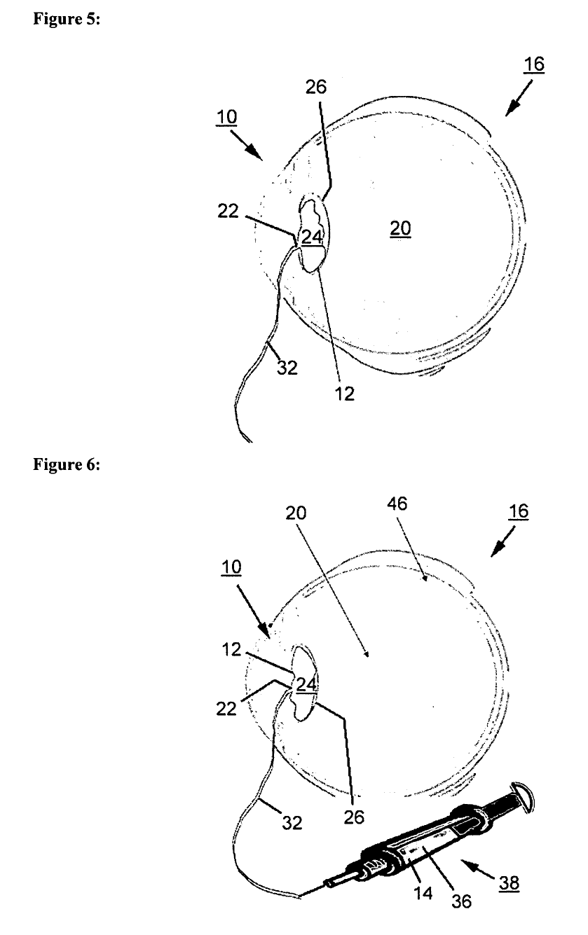

FIG. 5 shows a schematic representation of the eye implant 10 according to FIG. 4 upon introduction into the lenticular capsule 26 or the cavity 34 of the lenticular capsule 26, respectively. One recognizes that the hollow body 12 with its cavity 24 expands within the capsule bag 26.

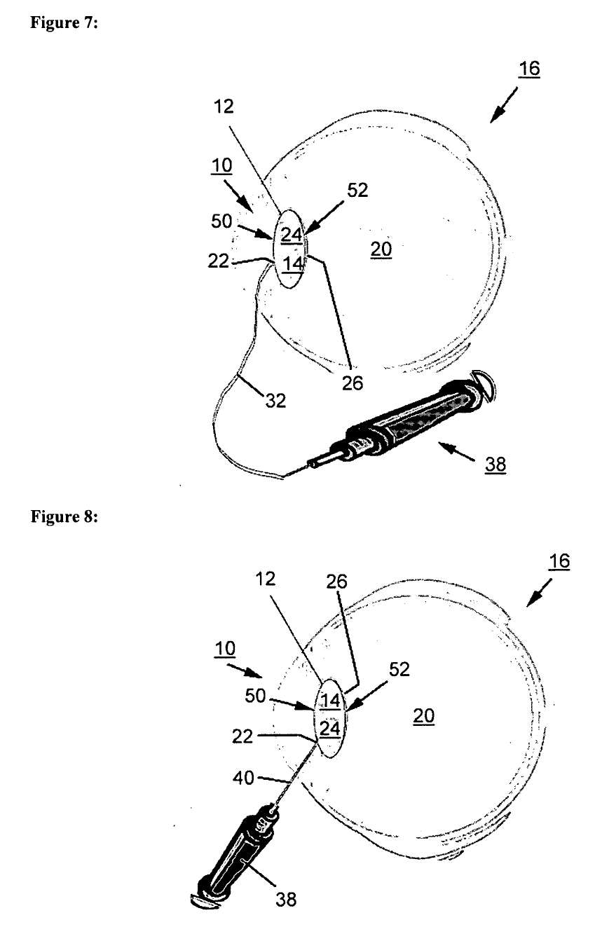

In FIG. 6, the eye implant 10 according to FIG. 4 is shown with connected filling device 38. One recognizes that the filling device 38 has a reservoir 36 with the medium 14. Then, filling the hollow body 12 of the eye implant 10 is effected through a corresponding connection of the filling device 38 to the tube connection 32 (compare FIG. 7). After completely filling the hollow body 12 or the cavity 24 of the hollow body 12 with the medium 14, respectively, the hollow body 12 approximately models the shape of the previously removed eye lens. In the filled state, the eye implant has a curved front wall 50 and a back wall 52 facing the vitreous body 20. After completely filling the eye implant 10, the tube connection 32 is detached from the hollow body 12. The valve device 22 provides for exit of the medium 14 into the surrounding eye tissue not to occur. The medium 14 can be liquid and/or semi-liquid and/or gaseous in the illustrated embodiment as well as also in the following embodiments. Other states of aggregation of the medium are also conceivable.

FIG. 8 shows a schematic representation of the eye implant 10 according to a second embodiment in a state filled with medium. One recognizes that instead of the tube connection 32, the medium 14 is now filled by means of a cannula 40 of a syringe-like filling device 38. Therein, the cannula 40 penetrates the valve device 22 of the hollow body 12 and thus places the medium 14 in the cavity 24 of the hollow body 12. After completely filling the cavity 24, the filling device 38 is retracted and the valve device 22 closes the hollow body 12. One recognizes that the eye implant 10 again has a curved front wall 50 and a curved back wall 52 facing the vitreous body 20 in the completely filled state.

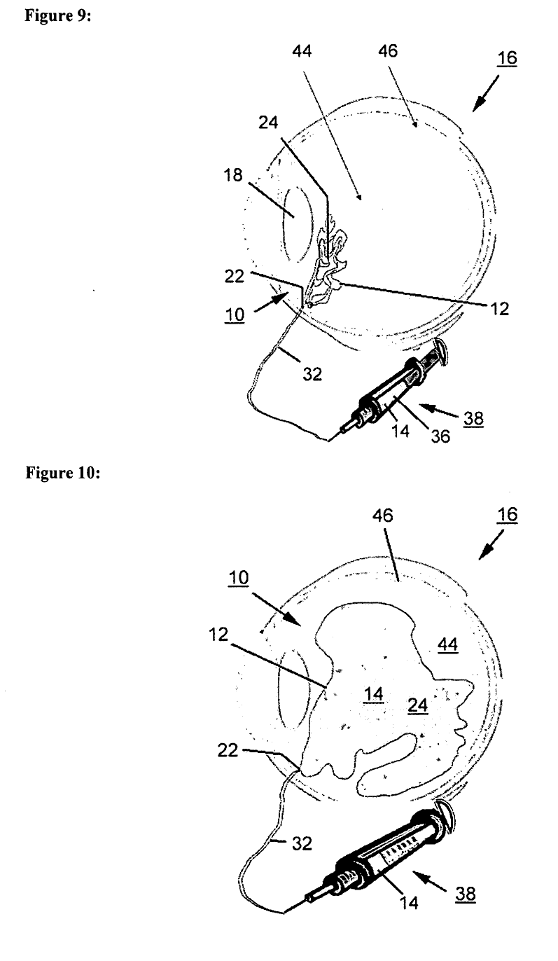

FIG. 9 shows a schematic representation of an eye implant 10 according to a third embodiment during introduction into a posterior orbit 44 of the human eye 16. The vitreous body 20 has been removed from the posterior orbit 44. The eye implant 10 consisting of the hollow boy 12 with the cavity 24 is introduced into the posterior orbit 44 through a small cut, through which the vitreous body 20 has already been removed. Subsequently, the medium 14 is filled into the cavity 24 of the hollow body 12 through the valve device 22, the tube connection 32 detachably connected thereto, and the filling device 38 connected to the tube connection 32 (compare FIG. 10).

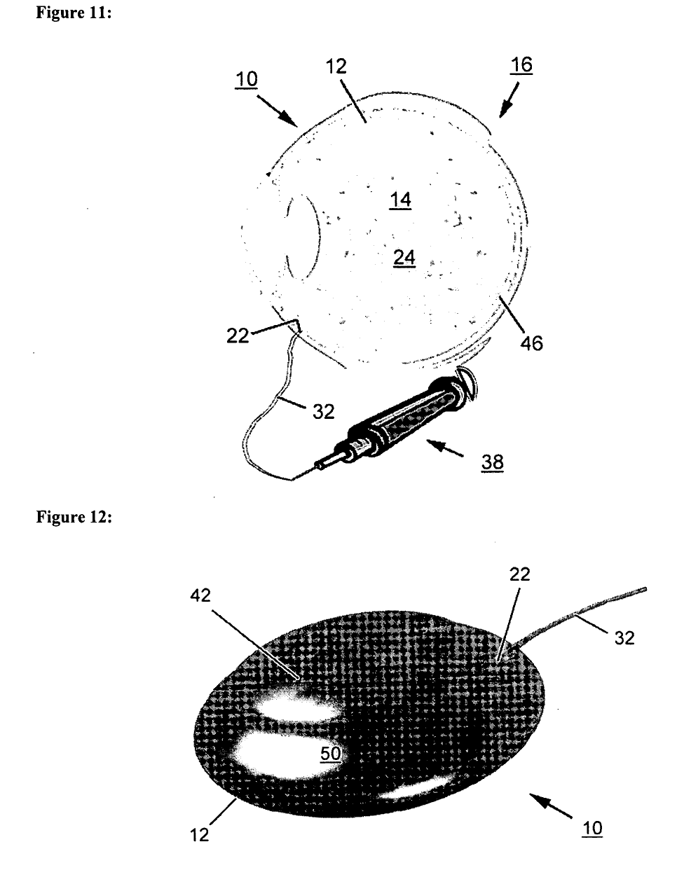

In state completely filled with the medium 14, illustrated in FIG. 11, the hollow body 12 approximately models the shape of the vitreous body 20 of the human or animal eye 16 and abuts the retina 46. After completely filling the hollow body 12, the tube connection 32 is detached from the eye implant 10 or the valve device 22 thereof, respectively.

FIG. 12 shows a representation of an eye implant 10 according to a fourth embodiment. One recognizes that the hollow body 12 has a device 42 having optical refractive power on its side 50 facing away from the interior of the eye. In the illustrated embodiment, it is an optical lens integrally formed with the hollow body 12. Further, one recognizes the tube connection 32 detachably connected to the hollow body 32 at the valve device 22. In the illustrated embodiment, the eye implant 10 serves as a substitute for an eye lens.

Although shown in only some embodiments in the figures, it will be recognized from the description set out herein that the present invention contemplates eye implants which, although not constructed exactly as shown in the figures, function in substantially similar fashion to achieve substantially similar results as the eye implants which are shown. All such changes are intended to fall within the spirit and scope of the following claims.

What is claimed is:

1. Artificial eye implant, especially as a substitute for an eye lens or a vitreous body of a human or animal eye, consisting of a hollow body, wherein said hollow body is made of a biocompatible, flexible and extensible material and is able to be filled with a medium.

2. Eye implant according to claim 1, wherein said hollow body is formed lens-shaped in a state filled with said medium.

3. Eye implant according to claim 2, wherein said hollow body has at least one valve device for filling said cavity with said medium.

4. Eye implant according to claim 2, wherein said hollow body has at least one tube connection for filling said medium into said cavity.

5. Eye implant according to claim 1 wherein said hollow body approximately models the shape of the vitreous body of the human or animal eye in a state filled with said medium.

6. Eye implant according to any claim 5, wherein said hollow body has at least one valve device for filling said cavity with said medium.

7. Eye implant according to claim 5, wherein said hollow body has at least one tube connection for filling said medium into said cavity.

8. Eye implant according to claim 1, wherein said hollow body has at least one device with optical refractive power on its side facing away from the interior of the eye.

9. Eye implant according to claim 1, wherein said hollow body has at least one device with optical refractive power on its side facing the interior of the eye.

10. Eye implant according to claim 1, wherein said medium is liquid and/or semi-liquid and/or gaseous.

11. A method of positioning an artificial eye implant within the eye of a human or an animal comprising the steps of:
a) preparing an eye to receive an intraocular artificial eye implant wherein said implant consists of a hollow body made of a biocompatible, flexible and extensible material, said hollow body is able to be filled with a medium;
b) inserting said intraocular eye implant into said eye via a small cut in said eye;
c) positioning said implant in the non-filled state in the capsule bag of the eye;
d) filling said implant with said medium; and
e) closing the eye where said implant was inserted.

12. The method of claim 9, wherein said preparing step is carried out by cutting and removing a lens of the human or animal eye.

13. The method of claim 9, wherein said filling step is carried out by filling said cavity via a valve device of said implant.

14. The method of claim 9, wherein said filling step is carried out by filling said cavity via at least one tube connection of said implant.

15. The method of claim 9, wherein said filling step is carried out by filling said cavity via a cannula of an external syringe-like filling device.

16. A method of positioning an artificial eye implant within the eye of a human or an animal comprising the steps of:
a) preparing an eye to receive an intraocular artificial eye implant wherein said implant consists of a hollow body made of a biocompatible, flexible and extensible material, said hollow body is able to be filled with a medium;
b) inserting said intraocular eye implant into said eye via a small cut in said eye;
c) positioning said implant in the non-filled state in a posterior orbit of the eye;
d) filling said implant with said medium; and
e) closing the eye where said implant was inserted.

17. The method of claim 16, wherein said preparing step is carried out by cutting and removing a vitreous body of the human or animal eye.

18. The method of claim 16, wherein said filling step is carried out by filling said cavity via a valve device of said implant.

19. The method of claim 16, wherein said filling step is carried out by filling said cavity via at least one tube connection of said implant.

20. The method of claim 16, wherein said filling step is carried out by filling said cavity via a cannula of an external syringe-like filling device.
